(19) United States
(12) Patent Application Publication
(10) Pub. No.: US 2005/0272732 A1
(43) Pub. Date: Dec. 8, 2005

(54) 5-ethyl-imidazo (5,1-F) (1,2,4,) triazin-4 (3h)-ones as phosphodiesterase inhibitors

(75) Inventors: Cristina Alonso-Alija, Haan (DE); Heike Gielen-Haertwig, Monheim (DE); Martin Michels, Solingen (DE); Dagmar Karthaus, Solingen (DE); Hilmar Bischoff, Wuppertal (DE); Nils Burkhardt, Velbert (DE); Volker Geiss, Ratingen (DE); Karl-Heinz Schlemmer, Wuppertal (DE); Nigel Cuthbert, Devon (DE); Mary F. Fitzgerald, Oxford (GB); Graham Sturton, Berkshire (GB); Ulrich Niewohner, Wermelskirchen (DE); Maria Niewohner, Wermelskirchen (DE)

Correspondence Address: JEFFREY M. GREENMAN, BAYER PHARMACEUTICALS CORPORATION, 400 MORGAN LANE, WEST HAVEN, CT, 06516, US

(73) Assignee: Bayer Healthcare AG, Leverkusen (DE)

(21) Appl. No.: 10/513,115

(22) PCT Filed: Apr. 22, 2003
(86) PCT No.: PCT/EP03/04140

§ 371 Date: May 25, 2005

(30) Foreign Application Priority Data
May 1, 2002 (GB) 0209989.3

(51) Int. Cl. A61K 31/53; C07D 487/04

(52) U.S. Cl. 514/243; 544/184

(57) ABSTRACT

The invention relates to novel 5-ethyl-imidazotriazinones, processes for their preparation and their use in medicaments, esp. for the treatment and/or prophylaxis of inflammatory processes and/or immune diseases.

The invention relates to novel 5-ethyl-imidazotriazinones, processes for their preparation and their use in medicaments, esp. for the treatment and/or prophylaxis of inflammatory processes and/or immune diseases.

Phosphodiesterases (PDEs) are a family of enzymes responsible for the metabolism of the intracellular second messengers cAMP (cyclic adenosine monophosphate) and cGMP (cyclic guanosine monophosphate). PDE 4, as a cAMP specific PDE, catalyses the conversion of cAMP to AMP and is the major if not sole isoform of the phosphodiesterase enzymes present in inflammatory and immune cell types. Inhibition of this enzyme leads to the accumulation of cAMP which, in these cells, leads to the inhibition of a range of pro-inflammatory functions. Uncontrolled production of inflammatory mediators can lead to acute and chronic inflammation, tissue damage, multi-organ failures and to death. Additionally, elevation of phagocyte cAMP leads to inhibition of oxygen radical production. This cell function is more sensitive than others such as aggregation or enzyme release.

It is now recognised that both asthma and COPD (Chronic obstructive pulmonary disease) are chronic inflammatory lung diseases. In the case of asthma the eosinophil is the predominant infiltrating cell. Subsequent release of superoxide radicals as well as damaging cationic proteins from these infiltrating cells are believed to play a role in the progression of the disease and development of airway hyperreactivity.

By contrast, in COPD the neutrophil is the predominant inflammatory cell type found in the lungs of sufferers. The action of mediators' and proteases released in the environment of the lung is believed to result in the irreversible airway obstruction seen in COPD. In particular the action of proteases in degrading the lung matrix results in fewer alveoli and is likely to be the major cause of accelerated long term lung function decline seen in this disease.

Treatment with a PDE 4 inhibitor is expected to reduce the inflammatory cell burden in the lung in both of these diseases [M. S. Barnette, “PDE 4 inhibitors in asthma and chronic obstructive pulmonary disease”, in: Progress in Drug Research, Birkhauser Verlag, Basel, 1999, pp. 193-229; H. J. Dyke and J. G. Montana, “The therapeutic potential of PDE 4 inhibitors”, Exp. Opin. Invest. Drugs 8, 1301-1325 (1999)].

WO 99/24433 and WO 99/67244 describe 2-phenyl-imidazotriazinones as synthetic intermediates for the synthesis of 2-(aminosulfonyl-phenyl)-imidazotriazinones as inhibitors of cGMP-metabolizing phosphodiesterases.

U.S. Pat. No. 4,278,673 discloses 2-aryl-imidazotriazinones with cAMP-phosphodiesterase inhibitory activity for the treatment of i.a. asthma

The present invention relates to compounds of the general formula (I) 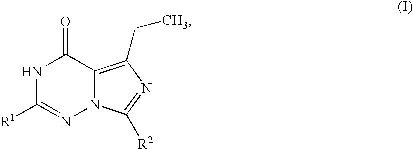
in which
R¹ denotes (C₅-C₈)-oxa-cyloalkyl, which can be substituted by 0, 1 or 2 residues independently selected from the group consisting of (C₁-C₆)-alkyl and (C₁-C₆)-alkoxy, or
   denotes (C₁-C₈)-alkyl or (C₃-Cg)-cycloalkyl, which are substituted by 1 or 2 identical or different (C₁-C₆)-alkoxy groups,
      and
R² denotes (C₃-C₈)-cycloalkyl, which can be substituted by 0, 1 or 2 identical or different (C₁-C₆)-alkyl groups.

The compounds according to this invention can also be present in the form of their salts, hydrates and/or solvates.

In general, salts with organic or inorganic bases or acids may be mentioned here.

Physiologically acceptable salts are preferred in the context of the present invention.

Physiologically acceptable salts can also be salts of the compounds according to this invention with inorganic or organic acids. Preferred salts are those with inorganic acids such as, for example, hydrochloric acid, hydrobromic acid, phosphoric acid or sulphuric acid, or salts with organic carboxylic or sulphonic acids such as, for example, acetic acid, maleic acid, fumaric acid, malic acid, citric acid, tartaric acid, ethane-sulphonic acid, benzenesulphonic acid, toluenesulphonic acid or naphthalenedisulphonic acid. Preferred pyridinium salts are salts in combination with halogen.

The compounds according to this invention can exist in stereoisomeric forms which either behave as image and mirror image (enantiomers), or which do not behave as image and mirror image (diastereomers). The invention relates both to the enantiomers and to the racemates, as well as the pure diastereomer and mixtures thereof. The racemates, like the diastereomers, can be separated into the stereoisomerically uniform constituents according to known methods.

Hydrates of the compounds of the invention are stoichiometric compositions of the compounds with water, such as for example hemi-, mono-, or dihydrates.

Solvates of the compounds of the invention or their salts are stoichiometric compositions of the compounds with solvents.

(C₁-C₆)-Alkoxy in general represents a straight chain or branched alkoxy residue with 1 to 6 carbon atoms. The following alkoxy residues are mentioned by way of example: methoxy, ethoxy, n-propoxy, isopropoxy, tert.-butoxy, n-pentoxy and n-hexoxy. Alkoxy residues with 1 to 4 carbon atoms are preferred. Alkoxy residues with 1 to 3 carbon atoms are especially preferred.

(C₁-C₈)-Alkyl in general represents straight chain or branched alkyl residues with 1 to 8, preferably 1 to 6 carbon atoms. The following alkyl residues are mentioned by way of example: methyl, ethyl, n-propyl, isopropyl, tert.-butyl, pentyl, hexyl, heptyl, octyl.

(C₃-C₈)-Cycloalkyl in general represents a cycloalkyl residue with 3 to 8 carbon atoms. The following cycloalkyl residues are mentioned by way of example: cyclopropyl, cyclobutyl, cyclopentyl, cyclohexyl, cycloheptyl, cyclooctyl. Cyclopentyl and cyclohexyl are preferred.

(C₅-C₈)-Oxa-cycloalkyl in general represents a saturated cyclic residue with 4 to 7 ring carbon atoms and 1 ring oxygen atom. The following oxa-cycloalkyl residues are preferred and mentioned by way of example: tetrahydrofuran-2-yl, tetrahydrofuran-3-yl, tetrahydropyran-2-yl, tetrahydropyran-3-yl, tetrahydropyran-4-yl, 2-oxa-cycloheptan-1-yl, 3-oxa-cycloheptan-1-yl, 4-oxa-cycloheptan-1-yl, 2-oxa-cyclooctan-1-yl, 3-oxa-cyclooctan-1-yl, 4-oxa-cyclooctan-1-yl. Especially preferred are tetrahydrofuranyl and tetrahydropyranyl.

Unless specified otherwise, when groups in compounds of the invention are optionally substituted, substitution by up to three identical or different residues is generally preferred.

A preferred embodiment of the invention relates to compounds of the general formula (I), in which
R¹ has the meaning indicated in claim 1, and
R² denotes 4-tert.-butylcyclohex-1-yl.

A preferred embodiment of the invention relates to compounds of the general formula (I), in which
R¹ has the meaning indicated in claim 1, and
R² denotes cis-4-tert.-butylcyclohex-1-yl.

The invention furthermore provides a process for preparing the compounds of the general formula (I) according to the invention, characterized in that compounds of the general formula (II) 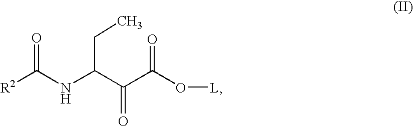
in which
R² is as defined above
   and
L represents straight-chain or branched alkyl having up to 4 carbon atoms,
are condensed with compounds of the general formula (III) 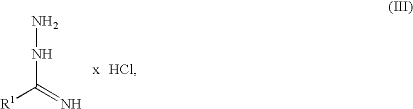
   in which
R¹ is as defined above,
preferably using ethanol as a solvent, to the compounds of the general formula (IV) 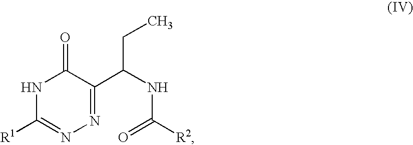
   in which R¹ and R² are as defined above,
which can optionally after isolation be reacted with a dehydrating agent, preferably phosphorous oxytrichloride, to yield the compounds of the general formula (I).

The compounds of the general formula (IV) can alternatively be prepared by
[A] condensation of compounds of the general formula (IIa) 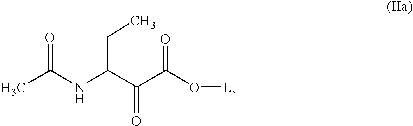
   in which
   L is as defined above,
   with compounds of the general formula (DI) to compounds of the general formula (IVa) 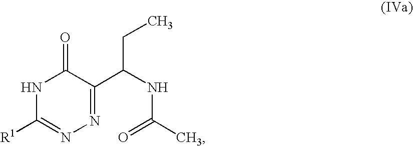
   in which
   R¹ is as defined above,
   preferably using ethanol as a solvent,
[B] followed by hydrolysis of the compounds of the general formula (IVa) to compounds of the general formula (V) 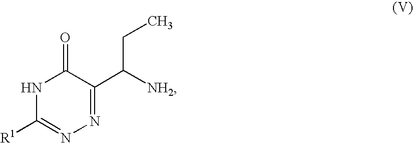
   in which
   R¹ is as defined above,
[C] and finally by condensation of the compounds of the general formula (V) with compounds of the general formula (VI) 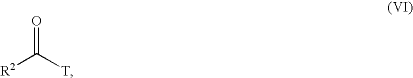
   in which
   R² is as defined above, and
   T represents a leaving group, preferably chlorine.

The process according to the invention can be illustrated using Me following scheme as an example:

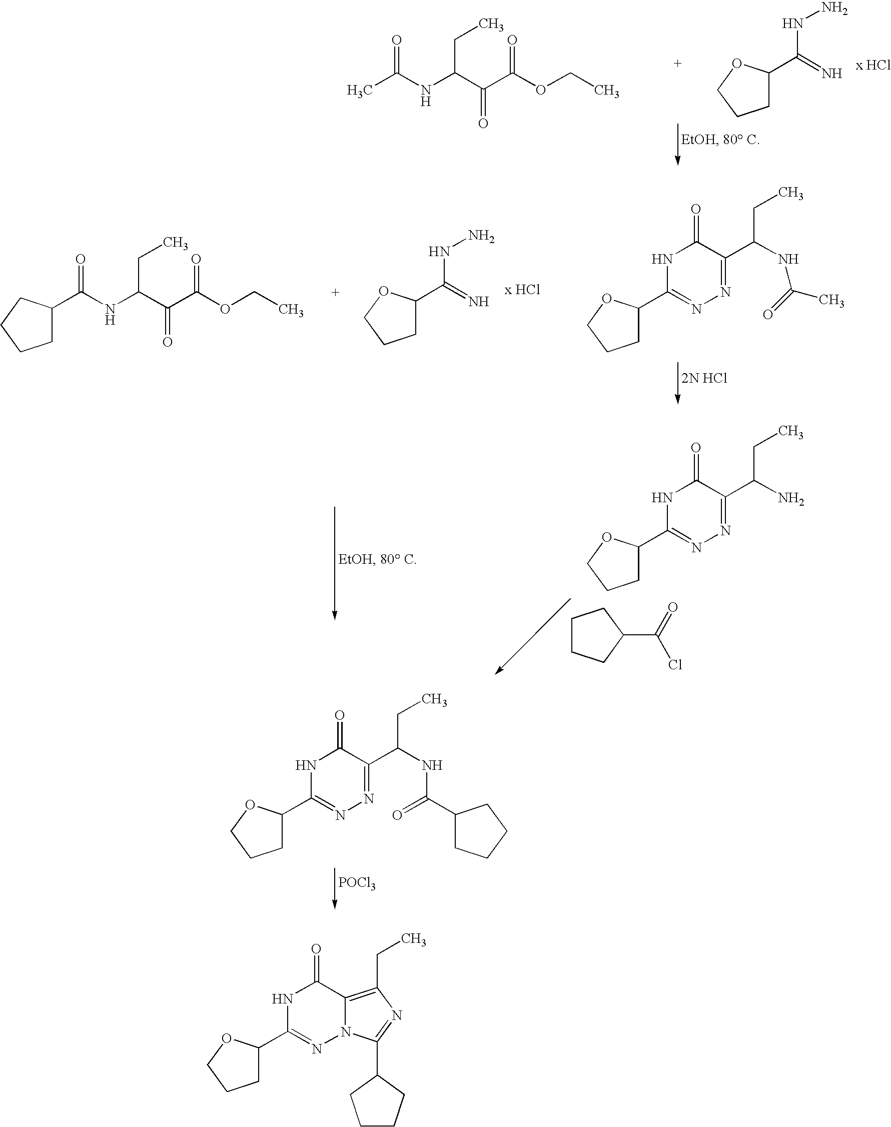

Solvents which are suitable for the individual steps are the customary organic solvents which do not change under the reaction conditions. These preferably include ethers, such as diethyl ether, dioxan, tetrahydrofuran, glycol dimethyl ether, or hydrocarbons, such as benzene, toluene, xylene, hexane, cyclohexane or mineral oil fractions, or halogenated hydrocarbons, such as dichloromethane, trichloromethane, carbon tetrachloride, dichloroethane, trichloroethylene or chlorobenzene, or ethyl acetate, dimethylformamide, dimethylsulfoxide, hexamethylphosphoric triamide, acetonitrile, acetone, or pyridine. It is also possible to use mixtures of the above-mentioned solvents. Particular preference is given to ethanol for the reaction (II)/(IIa)+(III)→(IV)/(IVa), and dichloroethane for the cyclisation (IV)→(I).

The reaction temperature can generally be varied within a relatively wide range. In general, the reaction is carried out in a range of from −20° C. to 200° C., preferably of from ° C. to 100° C.

The process steps according to the invention are generally carried out under atmospheric pressure. However, it is also possible to operate under superatmospheric pressure or under reduced pressure (for example, in a range from 0.5 to 5 bar).

The compounds of the general formula (IVa) are preferably hydrolysed to compounds of the general formula (V) under acidic conditions as for example in refluxing 2N hydrochloric acid.

The compounds of the general formula m are condensed with the compounds of the general formula (VI) to compounds of the general formula (IV) in inert solvents, if appropriate in the presence of a base.

Suitable inert solvents are the customary organic solvents which do not change under the reaction conditions. These preferably include ethers, such as diethyl ether, dioxan, tetrahydrofuran, glycol dimethyl ether, or hydrocarbons, such as benzene, toluene, xylene, hexane, cyclohexane or mineral oil fractions, or halogenated hydro-carbons, such as dichloromethane, trichloromethane, carbon tetrachloride, dichloroethylene, trichloroethylene or chlorobenzene, or ethyl acetate, dimethylformamide, dimethylsulfoxide, hexamethylphosphoric triamide, acetonitrile, acetone, or pyridine.

It is also possible to use mixtures of the above-mentioned solvents.

Suitable bases are generally alkali metal hydrides or alkali metal alkoxides, such as, for example, sodium hydride or potassium tert-butoxide, or cyclic amines, such as, for example, piperidine, pyridine, 4-N,N-dimethylaminopyridine or (C₁-C₄)-alkylamines, such as, for example, triethylamine. Preference is given to triethylamine, pyridine and/or 4-N,N-dimethylaminopyridine.

The base is generally employed in an amount of from 1 mol to 4 mol, preferably from 1.2 mol to 3 mol. in each case based on 1 mol of the compound of the formula (V).

The reaction temperature can generally be varied within a relatively wide range. In general, the reaction is carried out in a range of from −20° C. to 200° C., preferably of from 0° C. to 100° C.

Some of the compounds of the general formula (II) are known, or they are novel, and they can then be prepared by converting compounds of the general formula (VI)
R²—CO-T (VI),
in which
R² is as defined above
   and
T represents halogen, preferably chlorine,
   initially by reaction with α-amino-butyric acid in inert solvents, if appropriate in the presence of a base and trimethylsilyl chloride, into the compounds of the general formula (VII) 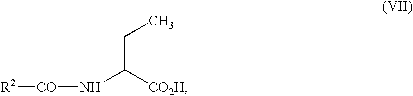
   in which
R² is as defined above,
and finally reacting with the compound of the formula (VIII) 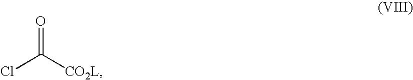
   in which
L is as defined above,
in inert solvents, if appropriate in the presence of a base.

The compounds of the general formula (IIa) can be prepared analogously.

Suitable solvents for the individual steps of the process are the customary organic solvents which do not change under the reaction conditions. These preferably include ethers, such as diethyl ether, dioxan, tetrahydrofuran, glycol dimethyl ether, or hydrocarbons, such as benzene, toluene, xylene, hexane, cyclohexane or mineral oil fractions, or halogenated hydrocarbons, such as dichloromethane, trichloromethane, carbon tetrachloride, dichloroethylene, trichloroethylene or chlorobenzene, or ethyl acetate, dimethylformamide, dimethylsulfoxide, hexamethylphosphoric triamide, acetonitrile, acetone, or pyridine. It is also possible to use mixtures of the above-mentioned solvents. Particular preference is given to dichloromethane for the first step and to a mixture of tetrahydrofuran and pyridine for the second step.

Suitable bases are generally alkali metal hydrides or alkali metal alkoxides, such as, for example, sodium hydride or potassium tert-butoxide, or cyclic amines, such as, for example, piperidine, pyridine, 4-N,N-dimethylaminopyridine or (C₁-C₄)-alkyl-amines, such as, for example, triethylamine. Preference is given to triethylamine, pyridine and/or 4-N,N-dimethylaminopyridine.

The base is generally employed in an amount of from 1 mol to 4 mol, preferably from 1.2 mol to 3 mol. in each case based on 1 mol of the compound of the formula (VII).

The reaction temperature can generally be varied within a relatively wide range. in general, the reaction is carried out in a range of from −20° C. to 200° C., preferably of from ° C. to 100° C.

The compounds of the general formulae (VI) and (VII) are known per se, or they can be prepared by customary methods.

The compounds of the general formula (E) are known or can be prepared by reacting compounds of the general formula (IX)
R¹—Y (IX),
in which
R¹ is as defined above, and
Y represents a cyano, carboxyl, methoxycarbonyl or ethoxycarbonyl group,
   with ammonium chloride in toluene and in the presence of trimethylaluminium in hexane in a temperature range of from −20° C. to room temperature, preferably at 0° C. and atmospheric pressure, and reacting the resulting amidine, if appropriate in situ, with hydrazine hydrate.

The compounds of the general formula (IX) are known per se, or they can be prepared by customary methods.

The compounds of the general formula (1) inhibit the PDE 4 resident in the membranes of human neutrophils. One measured functional consequence of this inhibition is inhibition of superoxide anion production by stimulated human neutrophils.

The compounds of the general formula (1) can therefore be employed in medicaments for the treatment of inflammatory processes, esp. acute and chronic inflammatory processes, and/or immune diseases.

The compounds according to the invention are preferably suitable for the treatment and prevention of inflammatory processes i.e. acute and chronic inflammatory processes, and/or immune diseases, such as emphysema, alveolitis, shock lung, all kinds of chronic obstructive pulmonary diseases (COPD), adult respiratory distress syndrome (ARDS), asthma, bronchitis, cystic fibrosis, eosinophilic granuloma, arteriosclerosis, arthrosis, inflammation of the gastrointestinal tract, myocarditis, bone resorption diseases, reperfusion injury, Crohn's disease, ulcerative colitis, systemic lupus erythematosus, type I diabetes mellitus, psoriasis, anaphylactoid purpura nephritis, chronic glomerulonephritis, inflammatory bowel disease, atopic dermatitis, other benign and malignant proliferative skin diseases, allergic rhinitis, allergic conjunctivitis, vernal conjunctivitis, arterial restenosis, sepsis and septic shock, toxic shock syndrome, grafts vs. host reaction, allograft rejection, treatment of cytokine-mediated chronic tissue degeneration, rheumatoid arthritis, arthritis, rheumatoid spondylitis, osteoarthritis, coronary insufficiency, myalgias, multiple sclerosis, malaria, AIDS, cachexia, prevention of tumor growth and tissue invasion, leukemia, depression, memory impairment and acute stroke. The compounds according to the invention are additionally suitable for reducing the damage to infarct tissue after-reoxygenation.

The active component can act systemically and/or locally. For this purpose, it can be applied in a suitable manner, for example orally, parenterally, pulmonally, nasally, sublingually, lingually, buccally, rectally, transdermally, conjunctivally, otically or as an implant.

For these application routes, the active component can be administered in suitable application forms.

Useful oral application forms include application forms which release the active component rapidly and/or in modified form, such as for example tablets (non-coated and coated tablets, for example with an enteric coating), capsules, sugar-coated tablets, granules, pellets, powders, emulsions, suspensions, solutions and aerosols.

Parenteral application can be carried out with avoidance of an absorption step (intravenously, intraarterially, intracardially, intraspinally or intralumbarly) or with inclusion of an absorption (intramuscularly, subcutaneously, intracutaneously, percutaneously or intraperitoneally). Useful parenteral application forms include injection and infusion preparations in the form of solutions, suspensions, emulsions, lyophilisates and sterile powders.

Forms suitable for other application routes include for example inhalatory pharmaceutical forms (including powder inhalers, nebulizers), nasal drops/solutions, sprays; tablets or capsules to be administered lingually, sublingually or buccally, suppositories, ear and eye preparations, vaginal capsules, aqueous suspensions (lotions, shake mixtures), lipophilic suspensions, ointments, creams, milk, pastes, dusting powders or implants.

The active components can be converted into the recited application forms in a manner known per se. This is carried out using inert non-toxic, pharmaceutically suitable excipients. These include inter alia carriers (for example microcrystalline cellulose), solvents (for example liquid polyethylene glycols), emulsifiers (for example sodium dodecyl sulphate), dispersing agents (for example polyvinyl-pyrrolidone), synthetic and natural biopolymers (for example albumin), stabilizers (for example antioxidants such as ascorbic acid), colorants (for example inorganic pigments such as iron oxides) or taste and/or odor corrigents.

Generally it has proved advantageous in the case of parenteral application to administer amounts of about 0.001 to 1 mg/kg and preferably about 0.01 to 0.5 mg/kg of body weight to achieve efficacious results. In the case of oral administration, the amount is about 0.001 to 50 mg/kg and preferably about 0.001 to 20 mg/kg of body weight.

In spite of this, it can be necessary in certain circumstances to depart from the amounts mentioned, namely as a function of body weight, application route, individual behaviour towards the active component, manner of preparation and time or interval at which application takes place. It can for instance be sufficient in some cases to use less than the aforementioned minimum amount, while in other cases the upper limit mentioned will have to be exceeded. In the case of the application of larger amounts, it can be advisable to divide them into a plurality of individual doses spread through the day.

The percentages in the tests and examples which follows are, unless otherwise stated, by weight; parts are by weight. Solvent ratios, dilution ratios and concentrations reported for liquid/liquid solutions are each based on the volume.

Test Descriptions

1. Preparation of Human PMN

Human PMN (polymorphonuclear neutrophil leucocytes) are readily purified from peripheral blood. Phosphodiesterase in these cells is predominantly located in the membrane fraction. Ihibitory potency of compounds against this preparation correlate well with the anti-inflammatory activity as measured by inhibiton of superoxide production.

Blood was taken from healthy subjects by venous puncture and neutrophils were purified by dextran sedimentation and density gradient centrifugation on Ficoll Histopaque and resuspended in the buffered medium.

2. Assay of Human PMN Phosphodiesterase

This was performed as a particulate fraction from human PMN essentially as described by Souness and Scott [Biochem. J. 291, 389-395 (1993)]. Particulate fractions were treated with sodium vanadate/glutathione as described by the authors to express the discrete stereospecific site on the phosphodiesterase enzyme. The prototypical PDE 4 inhibitor, rolipram, had an IC₅₀ value in the range 450 nM-1500 nM, thus defining this preparation as the so-called “low affinity” [L] form. The preparation examples had IC₅₀-values within the range of 5 nM-400 nM.

3. Inhibition of FMLP-Stimulated Production of Superoxide Radical Anions

Neutrophils (2.5×10⁵ ml⁻¹) were mixed with cytochrome C (1.2 mg/ml) in the wells of a microtitre plate. Compounds according to the invention were added in dimethyl sulphoxide (DMSO). Compound concentration ranged from 2.5 nM to 10 μM, the DMSO concentration was 0.1% v/v in all wells. After addition of cytochalasin b (5 μg×ml⁻) the plate was incubated for 5 min at 37° C. Neutrophils were then stimulated by addition of 4×10⁻⁸ M FMLP (N-Formyl-Met-Leu-Phe) and superoxide generation measured as superoxide dismutase inhibitable reduction of cytochrome C by monitoring the OD₅₅₀ in a Thermo-max microtitre plate spectrophotometer. Initial rates were calculated using a Softmax kinetic calculation programme. Blank wells contained 200 units of superoxide dismutase.

The inhibition of superoxide production was calculated as follows:

$$\frac{[ 1 - ( Rx - Rb ) ]}{( Ro - Rb )} \times 100$$

Rx=Rate of the well containing the compound according to the invention
Ro=Rate in the control well
Rb=Rate in the superoxide dismutase containing blank well
   4. Assay of binding to the rolipram binding site (PDE 4 high affinity site; “H-PDE 4 form”) in rat brain membranes:

The activity of compounds on the PDE 4 high affinity site (“H-PDE 4 form”) is readily measured by determining their potency for displacement of [³H]-rolipram from its binding site ii rat brain membranes. Activity at this site is believed to be a measure of side effect potential (e.g. stimulation of stomach acid secretion, nausea and emesis).

The rolipram binding site assay was performed essentially as described by Schneider et al. [Eur. J. Pharmacol. 127, 105-115 (1986)].

5. Lipopolysaccharide (LPS)-Induced Neutrophil Influx into Rat Lung

Intranasal administration of LPS to rats causes a marked influx of neutrophils into the lungs measurable by histological or biochemical (myeloperoxidase content of the cell pellet) analysis of the bronchoalveolar lavage fluid 24 h later. Rats were treated with test compound or vehicle administered by the oral route 1 h prior to and 6 h after administration of intranasal LPS. 24 hours later animals were euthanatized and their lungs lavaged with PBS (phosphate buffered saline). Neutrophil and total cell numbers were analysed.

6. Emetic Potential in the Marmoset

Vehicle or test compound was administered by the oral route to conscious marmosets. Animals were observed for emetic episodes or abnormal behaviour for 1 h post dosing. In some experiments, if no adverse response was seen, a separate group of animals was tested at ½ log dose higher until emesis or abnormal behaviour was observed. The highest dose at which no abnormal behavior or emetic episodes occurred was recorded as the NOEL.

Materials and Methods

LC-MS Method A

| LC-parameters: | solution A: acetonitrile | | | |
|---|---|---|---|---|
| | solution B: 0.3 g 30% HCl/L water | | | |
| | column oven 50° C.; | | | |
| | column Symmetry C18 2.1 × 150 mm | | | |
| gradient: | time [min] | % A | % B | flow [mL/min] |
| | 0 | 10 | 90 | 0.9 |
| | 3 | 90 | 10 | 1.2 |
| | 6 | 90 | 10 | 1.2 |

LC-MS Method B

| LC-parameters: | solution A: acetonitrile/0.1% formic acid | | | |
|---|---|---|---|---|
| | solution B: water/0.1% formic acid | | | |
| | column oven 40° C.; | | | |
| | column Symmetry C18 2.1 × 50 mm | | | |
| gradient: | time [min] | % A | % B | flow [mL/min] |
| | 0 | 10 | 90 | 0.5 |
| | 4 | 90 | 10 | 0.5 |
| | 6 | 90 | 10 | 0.5 |
| | 6.1 | 10 | 90 | 1.0 |
| | 7.5 | 10 | 90 | 0.5 |

GC-MS method A

| | |
|---|---|
| Column: | HP-5 30 m × 320 μm × 0.25 μm |
| Carrier Gas: | Helium |
| Mode: | Constant flow, initial flow: 1.5 mL/min |
| Oven ramp: | initial temp: 60° C. |
| | initial time: 1 min |
| | rate: 14° C./min up to 300° C., then 300° C. 2 min |

Unless specified otherwise, the following chromatographic conditions were applied: chromatography was performed on silica gel Si 60; for flash chromatography, the usual conditions were followed as described in Still, J. Org. Chem. 43, 2923 (1978); mixtures of dichloromethane and methanol or cyclohexane and ethylacetate were used as eluants. Unless specified otherwise, reactions were executed under an argon atmosphere and under anhydrous conditions.

### Abbreviations

HPLC=high performance liquid chromatography
MS=mass spectroscopy
NMR=nuclear magnetic resonance spectroscopy
LC-MS=liquid chromatography combined with mass spectroscopy
GC-MS=gas chromatography combined with mass spectroscopy
MeOH=methanol
DMF=dimethylformamide
DMSO=dimethylsulfoxide
THF=tetrahydrofuran
   Starting Materials

### EXAMPLE 1A

### 2-(Acetylamino)butanoic acid

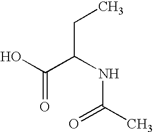

163 g (1.58 mol) 2-Aminobutanoic acid are dissolved in acetic acid, and 242 g (2.37 mol) acetic anhydride are added dropwise. The mixture is stirred for 2 h at 100° C. until completion of reaction, then the solution is evaporated to dryness in vacuo. The solid residue is suspended in ethyl acetate, filtered and washed with diethyl ether.

Yield: 220 g (95.9%)

¹H-NMR (CD₃OD): δ=0.97 (t, 3H), 1.65-1.93 (m, 2H), 1.99 (s, 3H), 4.29 (q, 1H) ppm.

### EXAMPLE 2A

### Ethyl 3-(acetylamino)-2-oxopentanoate

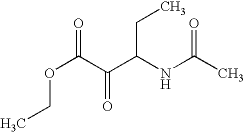

9.2 g (63.4 mmol) 2-(Acetylamino)butanoic acid are suspended in 120 ml tetrahydrofuran and heated to reflux together with 15.0 g (190 mmol) pyridine and a bit of N,N-dimethylaminopyridine. While heating at reflux, 17.3 g (127 mmol) ethyl chloro-(oxo)acetate are added dropwise. The reaction mixture is heated at reflux until no more evolution of gas can be observed. After cooling down to room temperature, the reaction mixture is added to ice water and the organic phase extracted with ethyl acetate. The dried organic phase is evaporated to dryness in vacuo, dissolved in ethanol and the solution directly used for the next reaction.

### EXAMPLE 3A

### Tetrahydro-2-furancarboximidamide hydrochloride

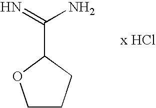

41.1 g (768 mmol, 5 equiv.) ammonium chloride are suspended in 400 ml of dry toluene under an argon atmosphere, and the mixture is cooled to 0° C. 385 ml (768 mmol, 5 equiv.) of a 2 M solution of trimethylaluminium in hexane are added dropwise, and the reaction mixture is stirred at room temperature until no more evolution of gas is observed. After addition of 20 g (154 mmol, 1 equiv.) methyl tetrahydro-2-furancarboxylate, the mixture is stirred at 80° C. bath temperature over night. It is then cooled down to 0° C., and 200 ml of methanol are added with consequent stirring for 1 hour at room temperature. After filtration, the solid is washed with methanol for several times and the solution is evaporated to dryness in vacuo.

Yield: 15.9 g (69%)

### EXAMPLE 4A

### 2-Methoxy-2-methylpropanimidamide hydrochloride

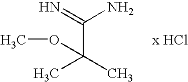

In analogy to the procedure for Example 3A, 20.0 g (202 mmol) 2-methoxy-2-methylpropanenitrile and proportionate amounts of the other reagents are used.

Yield: 24 g (78%)

MS (DCI/NH₃): m/z-117 [M+H]⁺

### EXAMPLE 5A

### Tetrahydro-2H-pyran-4-carboximidamide hydrochloride

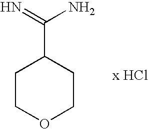

In analogy to the procedure for Example 3A, 20.0 g (139 mmol) methyl tetrahydro-2H-pyran-4-carboxylate and proportionate amounts of the other reagents are used.

Yield: 20 g (88%)

¹H-NMR (300 MHz, DMSO-d₆): δ=1.64-1.85 (m, 4H), 2.71-2.84 (m, 1H), 3.23-3.38 (m, 2H), 3.88-3.97 (m, 2H), 8.88-9.08 (m, 3H, NH) ppm.

### EXAMPLE 6A

### 1-Methoxycyclopropanecarboximidamide hydrochloride

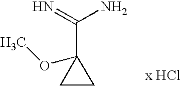

In analogy to the procedure for Example 3A, 4.30 g (29.8 mmol) ethyl 1-methoxy-cyclopropanecarboxylate and proportionate amounts of the other reagents are used.

Yield: 3.91 g (870%)

### EXAMPLE 7A

### rac-N-[1-(5-Oxo-3-tetrahydro-2-furanyl-4,5-dihydro-1,2,4-triazin-6-yl)propyl]acetamide

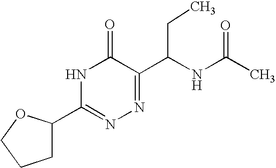

15.9 g (106 mmol, 1 equiv.) Tetrahydro-2-furancarboximidamide hydrochloride are suspended in 300 ml of ethanol and 6.34 g (127 mmol, 1.2 equiv.) hydrazine hydrate are added. After stirring at room temperature for 1 hour, 31.9 g (158 mmol, 1.5 equiv.) of the compound of Example 2A, dissolved in 30 ml of ethanol, are added. The reaction mixture is stirred at 80° C. (bath temperature) for 4 hours and then at room temperature over night. The mixture is evaporated to dryness in vacuo and the product is purified by chromatography (flash or column chromatography or preparative HPLC).

Yield: 8.61 g (27%)

¹H-NMR (200 MHz, CD₃OD, diastereomeric mixture): δ=1.18 (t, 3H), 2.02-2.75 (m, 9H, s at 2.17), 4.07-4.34 (m, 2H), 4.94-5.05 (m, 1H), 5.13-5.25 (m, 1H) ppm.

### EXAMPLE 8A

### N-{1-[3-(1-Methoxy-1-methylethyl)-5-oxo-4,5-dihydro-1,2,4-triazin-6-yl]propyl}-acetamide

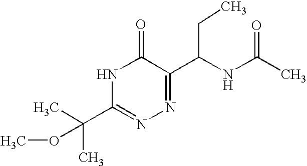

In analogy to the procedure for Example 7A, 10.0 g (65.5 mmol) 2-methoxy-2-methylpropanimidamide hydrochloride and proportionate amounts of the other reagents are used.

Yield: 11.9 g (61%)

¹H-NMR (300 MHz, CD₃OD): δ=0.98 (t, 3H), 1.51 (s, 6H), 1.63-2.02 (m, 5H, s at 1.98), 3.32 (s, 3H), 4.96-5.03 (m, 1H) ppm.

### EXAMPLE 9A

### N-[1-(5-Oxo-3-tetrahydro-2H-pyran-4-yl-4,5-dihydro-1,2,4-triazin-6-yl)propyl]-acetamide

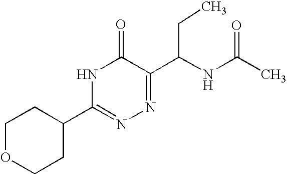

In analogy to the procedure for Example 7A, 10.0 g (60.7 mmol) tetrahydro-2H-pyran-4-carboximidamide hydrochloride and proportionate amounts of the other reagents are used.

Yield: 11.7 g (69%)

### EXAMPLE 10A

### N-{1-[3-(1-Methoxycyclopropyl)-5-oxo-4,5-dihydro-1,2,4-triazin-6-yl]propyl}-acetamide

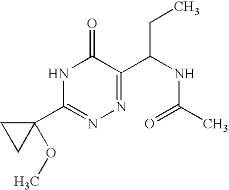

In analogy to the procedure for Example 7A, 4.00 g (26.6 mmol) methoxycyclopropanecarboximidamide hydrochloride and proportionate amounts of the other reagents are used.

Yield: 3.4 g (45%)

¹H-NMR (400 MHz, CD₃OD): δ 0.99 (t, 3H), 1.32-1.46 (m, 4H), 1.63-2.02 (m, 5H, s at 1.98), 3.39 (s, 3H), 4.96-5.01 (m, 1H) ppm.

### EXAMPLE 11A

### rac-6-(1-Aminopropyl)-3-tetrahydro-2-furanyl-1,2,4-triazin-5 (4H)-one

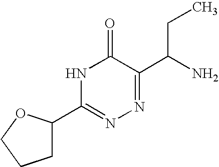

4 g (15.0 mmol, 1 equiv.) of N-[1-(5-Oxo-3-tetrahydro-2-furanyl-4,5-dihydro-1,2,4-triazin-6-yl)propyl]acetamide (Example 7A) are heated to reflux in 100 ml 2 N hydrochloric acid for 2 hours. After cooling down to room temperature, the mixture is neutralized with 10% sodium hydroxide and, after addition of ethanol, evaporated to dryness in vacuo. The residue is treated with methanol and the filtrate separated from the salts. The filtrate is evaporated to dryness in vacuo and the product purified by chromatography (flash or column chromatography or preparative HPLC).

Yield: 1.77 g (52%)

### EXAMPLE 12A

### 6-(1-Aminopropyl)-3-(1-methoxy-1-methylethyl)-1,2,4-triazin-5 (4H)-one

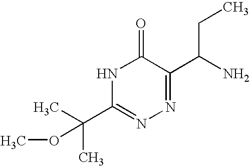

In analogy to the procedure for Example 11A, 6.00 g (22.4 mmol) N-{1-[3-(1-methoxy-1-methylethyl)-5-oxo-4,5-dihydro-1,2,4-triazin-6-yl]propyl} acetamide and proportionate amounts of the other reagents are used.

Yield: 2.8 g (55%)

¹H-NMR (300 MHz, CD₃OD): δ=0.97 (t, 3H), 1.52 (s, 6H), 1.78-2.10 (m, 2H), 3.23 (s, 3H), 4.21-4.26 (t, 1H) ppm.

### EXAMPLE 13A

### 6-(1-Aminopropyl)-3-tetrahydro-2H-pyran-4-yl-1,2,4-triazin-5 (4H)-one

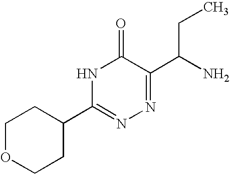

In analogy to the procedure for Example 11A, 11.7 g (41.7 mmol) N-[1-(5-oxo-3-tetrahydro-2H-pyran-4-yl-4,5-dihydro-1,2,4-triazin-6-yl)propyl]acetamide and proportionate amounts of the other reagents are used.

Yield: 7.4 g (74%)

¹H-NMR (300 MHz, CD₃OD): δ 0.98 (t, 3H), 1.74-2.15 (m, 6H), 2.79-2.90 (m, 1H), 3.46-3.57 (m, 2H), 3.97-4.06 (m, 2H), 4.37 (t, 1H) ppm.

### EXAMPLE 14A

### 6-(1-Aminopropyl)-3-(1-methoxycyclopropyl)-1,2,4-triazin-5 (4H)-one

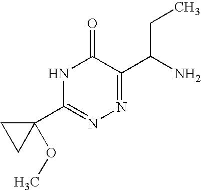

In analogy to the procedure for Example 11A, 3.43 g (12.9 mmol) N-{1-[3-(1-methoxycyclopropyl)-5-oxo-4,5-dihydro-1,2,4-triazin-6-yl]propyl}acetamide and proportionate amounts of the other reagents are used.

Yield: 1.33 g (46%)

¹H-NMR (300 MHz, CD₃OD): δ=1.00 (t, 3H), 1.24-1.41 (m, 4H), 1.82-2.14 (m, 2H), 3.43 (s, 3H), 4.26-4.32 (t, 1H) ppm.

### EXAMPLE 15A

### N-[1-(5-Oxo-3-tetrahydro-2-furanyl-4,5-dihydro-1,2,4-triazin-6-yl)propyl]cyclopentanecarboxamide

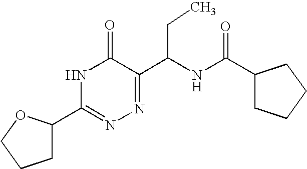

890 mg (3.97 mmol, 1 equiv.) 6-(1-Aminopropyl)-3-tetrahydro-2-furanyl-1,2,4-triazin-5 (4H)-one (Example 11A) are suspended in 10 ml dichloromethane, 482 mg (4.76 mmol, 1.2 equiv.) triethylamine and 526 mg (3.97 mmol, 1 equiv.) cyclopentanecarbonyl chloride are added. The reaction mixture is stirred at room temperature until completion of reaction (1-2 hours). The crude product is used in the next step without further purification.

### EXAMPLE 16A

### 4-cis-tert-Butyl-N-[1-(5-oxo-3-tetrahydro-2-furanyl-4,5-dihydro-1,2,4-triazin-6-yl)-propyl]cyclohexanecarboxamide

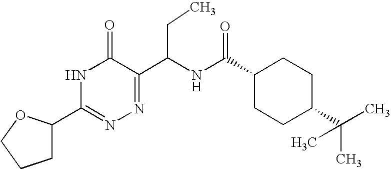

In analogy to the procedure for Example 15A, 860 mg (3.84 mmol, 1 equiv.) 6-(1-aminopropyl)-3-tetrahydro-2-furanyl-1,2,4-triazin-5 (4H)-one, 777 mg (3.84 mmol, 1 equiv.) cis-4-tert-butylcyclohexanecarbonyl chloride (Example 26A) and proportionate amounts of the other reagents are used.

### EXAMPLE 17A

### 4-trans-tert-Butyl-N-{1-[3-(1-methoxy-1-methylethyl)-5-oxo-4,5-dihydro-1,2,4-triazin-6-yl]propyl} cyclohexanecarboxamide

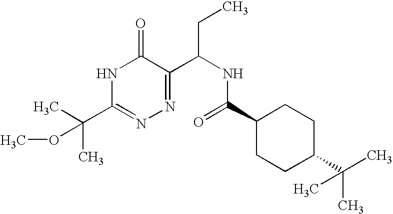

In analogy to the procedure for Example 15A, 200 mg (0.88 mmol) 6-(1-aminopropyl)-3-(1-methoxy-1-methylethyl)-1,2,4-triazin-5 (4H)-one, 179 mg (0.88 mmol) trans-tert-butylcyclohexanecarbonyl chloride (Example 27A) and proportionate amounts of the other reagents are used.

### EXAMPLE 18A

### 4-cis-tert-Butyl-N-{1-[3-(1-methoxy-1-methylethyl)-5-oxo-4,5-dihydro-1,2,4-triazin-6-yl]propyl} cyclohexanecarboxamide

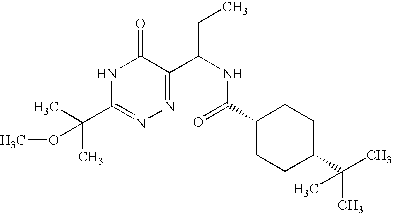

In analogy to the procedure for Example 15A, 800 mg (3.54 mmol) 6-(1-amino-propyl)-3-(1-methoxy-1-methylethyl)-1,2,4-triazin-5 (4H)-one, 717 mg (3.54 mmol) cis-4-tert-butylcyclohexanecarbonyl chloride (Example 26A) and proportionate amounts of the other reagents are used.

### EXAMPLE 19A

### N-{1-[3-(1-Methoxy-1-methylethyl)-5-oxo-4,5-dihydro-1,2,4-triazin-6-yl]propyl}-4-methylcyclohexanecarboxamaide

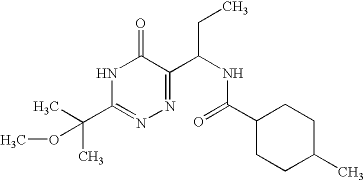

In analogy to the procedure for Example 15A, 700 mg (3.09 mmol) 6-(1-amino-propyl)-3-(1-methoxy-1-methylethyl)-1,2,4-triazin-5 (4H)-one, 497 mg (3.09 mmol) cis/trans-4-methylcyclohexanecarbonyl chloride (Example 28A) and proportionate amounts of the other reagents are used.

### EXAMPLE 20A

### N-{1-[3-(1-Methoxy-1-methylethyl)-5-oxo-4,5-dihydro-1,2,4-triazin-6yl]propyl}-cyclopentanecarboxamide

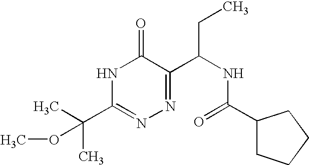

In analogy to the procedure for Example 15A, 800 mg (3.54 mmol) 6-(1-amino-propyl)-3-(1-methoxy-1-methylethyl)-1,2,4-triazin-5 (4H)-one, 800 mg (3.54 mmol) cyclopentanecarbonyl chloride and proportionate amounts of the other reagents are used.

### EXAMPLE 21A

### 4-cis-tert-Butyl-N-[1-(5-oxo-3-tetrahydro-2H-pyran-4-yl-4,5-dihydro-1,2,4-triazin-6-yl)propyl]cyclohexanecarboxamide

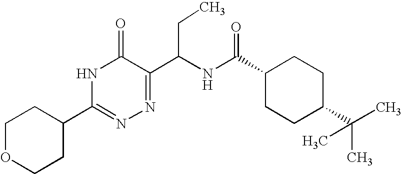

In analogy to the procedure for Example 15A, 1.0 g (4.20 mmol) 6-(1-aminopropyl)-3-tetrahydro-2H-pyran-4-yl-1,2,4-triazin-5 (4H)-one, 851 mg (4.20 mmol) cis-4-tert-butylcyclohexanecarbonyl chloride (Example 26A) and proportionate amounts of the other reagents are used.

### EXAMPLE 22A

### N-[1-(5-Oxo-3-tetrahydro-2H-pyran-4-yl-4,5-dihydro-1,2,4-triazin-6yl)propyl]cyclopentanecarboxamide

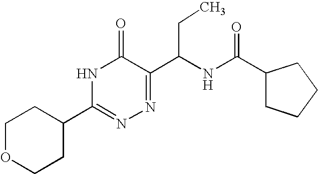

In analogy to the procedure for Example 15A, 1.0 g (4.20 mmol) 6-(1-aminopropyl)-3-tetrahydro-2H-pyran-4-yl-1,2,4-triazin-5 (4H)-one, 556 mg (4.20 mmol) cyclopentanecarbonyl chloride and proportionate amounts of the other reagents are used

### EXAMPLE 23A

### N-{1-[3-(1-Methoxycyclopropyl)-5-oxo-4,5-dihydro-1,2,4-triazin-6-yl]propyl}cyclopentanecarboxamide

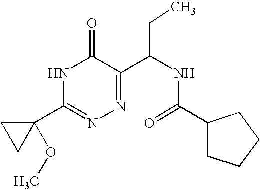

In analogy to the procedure for Example 15A, 200 mg (0.89 mmol) 6-(1-amino-propyl)-3-(1-methoxycyclopropyl)-1,2,4-triazin-5(4H)-one, 118 mg (0.89 mmol) cyclopentanecarbonyl chloride and proportionate amounts of the other reagents are used.

### EXAMPLE 24A

### 4-tert-Butyl-N-{-[3-(1-methoxycyclopropyl)-5-oxo-4,5-dihydro-1,2,4-triazin-6-yl]-propyl}cyclohexanecarboxamide

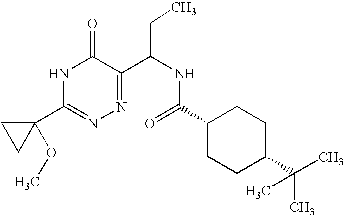

In analogy to the procedure for Example 15A, 200 mg (0.89 mmol) 6-(1-amino-propyl)-3-(1-methoxycyclopropyl)-1,2,4-triazin-5 (4H)-one, 181 mg (0.89 mmol) cis-4-tert-butylcyclohexanecarbonyl chloride (Example 26A) and proportionate amounts of the other reagents are used.

### EXAMPLE 25A

### cis- and trans-4-tert-Butylcyclohexanecarboxylic acid

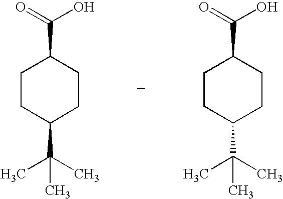

A preparative HPLC separation of cis- and trans-4-tert-butylcyclohexanecarboxylic acid was carried out under the following conditions:

| | |
|---|---|
| Feed: | 10 g isomeric mixture of cis- and trans-4-tert-butyl- |
| | cyclo-hexanecarboxylic acid dissolved in 500 ml |
| | iso-hexane (80%)/tert-butylmethylether (20%) |
| Column: | 330 × 100 mm; Self Packing Device NW 100; Merck |
| Stationary phase: | LiChrospher Si 60, 12 μm, Merck |
| Mobile phase: | iso-hexane/tert-butylmethylether (4/1 v/v) + 0.25 |
| | vol-% acetic acid |
| Flow: | 150 ml/min |
| Injection volume: | 70 ml (= 1.4 g compound) |
| Wave length: | 210 nm |
| Temperature: | 25° C. |

The sample run on this column was repeatedly injected every 30 minutes. The cis-isomer is the first eluting compound.
cis-isomer:

mp.: 118° C.

¹H-NMR (300 MHz, DMSO): δ 0.9 (t, 3H), 1.0 (m, 3H), 1.4 (m, 2H), 1.6 (m, 1H), 2.1 (m, 2H), 2.5 (m, 1H), 12.0 (s, 1H) ppm.
trans-isomer:

mp.: 172° C.

¹H-NMR (300 MHz, DMSO): δ=0.9 (t, 3H), 1.0 (m, 3H), 1.3 (m, 2H), 1.7 (m, 1H), 1.9 (m, 2H), 2.1 (m, 1H), 11.9 (s, 1H) ppm.

### EXAMPLE 26A

### cis-4-tert-Butylcyclohexanecarbonyl chloride

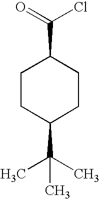

2.0 g (10.85 mmol) cis-4-tert-Butylcyclohexanecarboxylic acid are dissolved in 50 ml dichloromethane, 1.65 g (1.3.02 mmol) ethanedioyl dichloride are added and the solution is stirred at room temperature for one hour. The mixture is then stirred at reflux for two hours and, after cooling down to room temperature, evaporated to dryness in vacuo. The residue is then dissolved in toluene two times and again evaporated to dryness in vacuo. The residue is used in the next step without further purification.

### EXAMPLE 27A

### trans-4-tert-Butylcyclohexanecarbonyl chloride

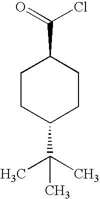

11.0 g (59.7 mmol) trans-4-tert-Butylcyclohexanecarboxylic acid are dissolved in 400 ml dichloromethane plus a few drops of DMF, 9.09 g (71.6 mmol) ethanedioyl dichloride are added and the solution is stirred at room temperature for one hour. The mixture is then stirred at reflux for two hours and, after cooling down to room temperature, evaporated to dryness in vacuo. The residue is then dissolved in toluene two times and again evaporated to dryness in vacuo. The residue is used in the next step without further purification.

### EXAMPLE 28A

### cis/trans-4-Methylcyclohexanecarbonyl chloride

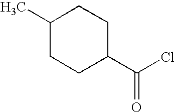

5.0 g (35.2 mmol) cis/trans-4-Methylcyclohexanecarboxylic acid are dissolved in 30 ml dichloromethane plus a few drops of dimethylformamide. 5.36 g (42.2 mmol) ethanedioyl dichloride in 5 ml dichloromethane are added dropwise, and the solution is stirred at room temperature for one hour, followed by additional stirring at reflux for two hours. The solvent is then removed in vacuo, the residue is dissolved in toluene and again evaporated to dryness. The residue is used in the next step without further purification.

### PREPARATION EXAMPLES

### EXAMPLE 1

### 7-Cyclopentyl-5-ethyl-2-tetrahydro-2-furanylimidazo[5,1-f][1,2,4]triazin-4 (3H)-one

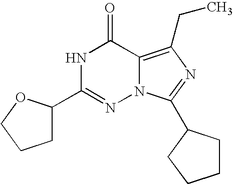

1.27 g (3.96 mmol, 1 equiv.) crude N-[1-(5-oxo-3-tetrahydro-2-furanyl-4,5-dihydro-1,2,4-triazin-6-yl)propyl]cyclopentanecarboxamide (Example 15A) are suspended in 20 ml dichloroethane, and 0.91 g (5.94 mmol, 1.5 equiv.) phosphoroxychloride are added. The mixture is stirred at reflux for 3 hours. After cooling down to ice bath temperature, saturated aqueous NaHCO₃ is added. The mixture is then evaporated to dryness in vacuo. The product is purified by chromatography (flash or column chromatography) and additional enantiomer separation on a chiral silica gel phase. A particularly suitable, commercially available chiral polyamide silica gel phase (CSP) for the separation of the enantiomers is Chiralcel OD with iso-hexane/iso-propanol mixtures as eluent.

| | | |
|---|---|---|
| Yield: | racemic mixture: | 610 mg (51%) |
| | enantiomer A: | 97 mg (8.1%) |
| | enantiomer B: | 170 mg (14%) |

¹H-NMR (400 MHz, CD₃OD): δ=1.25 (t, 3H), 1.66-1.78 (m, 2H), 1.82-1.96 (m, 4H), 1.98-2.13 (m, 4H), 2.20-2.35 (m, 2H), 2.93 (q, 2H), 3.53-3.63 (m, 1H), 3.88-3.96 (m, 1H), 4.024.09 (m, 1H), 4.74 (t, 1.H) ppm.

Specific optical rotation (solvent methanol): enantiomer A: +3.9° (c=0.5195 g/100 ml) enantiomer B: −11.1° (c=0.4920 g/100 ml)

### EXAMPLE 2

### cis-7-(4-tert-Butylcyclohexyl)-5-ethyl-2-tetrahydro-2-furanylimidazo[5,1-f][1,2,4]-triazin-4 (3H)-one

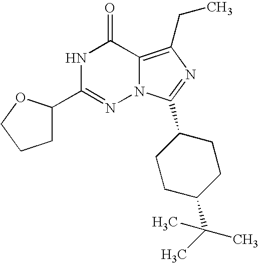

In analogy to the procedure for Example 1, 1.5 g (3.84 mmol) crude cis-4-tert-butyl-1-methyl-N-[1-(5-oxo-3-tetrahydro-2-furanyl-4,5-dihydro-1,2,4-triazin-6-yl)propyl]-cyclohexanecarboxamide and 589 mg (3.84 mmol) phosphoric trichloride are stirred at reflux for 3 hours, proportionate amounts of the solvents are used.

| | | |
|---|---|---|
| Yield: | racemic mixture: | 490 mg (34%) |
| | enantiomer A: | 112 mg (7.8%) |
| | enantiomer B: | 136 mg (9.5%) |

¹H-NMR (400 MHz, CD₃OD): δ=0.85 (s, 9H), 1.08-1.17 (m, 1H), 1.26 (t, 3H), 1.42-1.78 (m, 6H), 1.97-2.09 (m, 2H), 2.19-2.38 (m, 4H), 2.95 (q, 2H), 3.43-3.48 (m, 1H), 3.88-3.95 (m, 1H), 4.01-4.09 (m, 1H), 4.73 (t, 11) ppm.

Specific optical rotation (solvent methanol): enantiomer A: +0.7° (c=0.5240 g/100 ml) enantiomer B: −6.9° (c 0.5455 g/100 ml)

### EXAMPLE 3

### trans-7-(4-tert-Butylcyclohexyl)-5-ethyl-2-(1-methoxy-1-methylethyl)imidazo[5,1f]-[1,2,4]triazin-4 (3H)-one

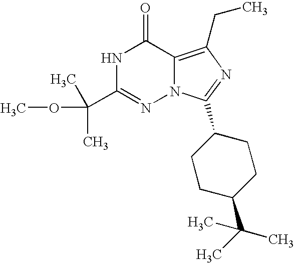

In analogy to the procedure for Example 1, 347 mg (0.88 mmol) crude trans-4-tert-butyl-N-{1-[3-(1-methoxy-1-methylethyl)-5-oxo-4,5-dihydro-1,2,4-triazin-6-yl]-propyl}-1-methylcyclohexanecarboxamide and 271 mg (1.77 mmol) phosphoric trichloride are stirred at reflux for 3 hours, proportionate amounts of the solvents are used.

Yield: 202 mg (61%)

¹H-NMR (300 MHz, CD₃OD): 8-0.91 (s, 9H), 1.15-1.20 (m, 1H), 1.25 (t, 3H), 1.55 (s, 6H), 1.65-2.06 (m, 8H), 2.93 (q, 2H), 3507-3.18 (m, 1H), 3.21 (s, 3H) ppm.

### EXAMPLE 4

### cis-7-(4-tert-Butylcyclohexyl)-5-ethyl-2-tetrahydro-2H-pyran-4-ylimidazo[5,1f]-[1,2,4]triazin-4(311)-one

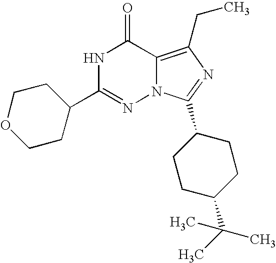

In analogy to the procedure for Example 1, 1.70 g (4.20 mmol) crude cis-4-tert-butyl-1-methyl-N-[1-(5-oxo-3-tetrahydro-2H-pyran-4-yl-4,5-dihydro-1,2,4-triazin-6-yl)-propyl]cyclohexanecarboxamide and 965 mg (6.30 mmol) phosphoric trichloride are stirred at reflux for 3 hours, proportionate amounts of the solvents are used.

Yield: 825 mg (51%)

¹H-NMR (400 MHz, CD₃OD): δ=0.84 (s, 9H), 1.08-1.17 (m, 1H), 1.25 (t, 3H), 1.42-1.55 (m, 2H), 1.58-1.65 (m, 2H), 1.67-1.77 (m, 2H), 1.80-1.93 (m, 4H), 2.32-2.40 (m, 2H), 2.72-2.81 (m, 1H), 2.94 (q, 2H), 3.43-3.54 (m, 3H), 3.99-4.05 (m, 2H) ppm.

### EXAMPLE 5

### 7-Cyclopentyl-5-ethyl-2-tetrahydro-2H-pyran-4-ylimidazo[5,1-f][1,2,4]triazin-4(3H)-one

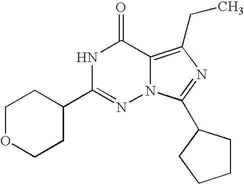

In analogy to the procedure for Example 1, 1.40 g (4.19 mmol) crude N-[1-(5-oxo-3-tetrahydro-2H-pyran-4-yl-4,5-dihydro-1,2,4-triazin-6-yl)propyl]cyclopentanecarboxamide and 964 mg (6.29 mmol) phosphoric trichloride are stirred at reflux for 3 hours, proportionate amounts of the solvents are used.

Yield: 846 mg (64%)

¹H-NMR (500 MHz, CD₃OD): δ=1.25 (t, 3H), 1.66-1.79 (m, 2H), 1.81-1.96 (m, 8H), 2.01-2.13 (m, 2H), 2.72-2.80 (m, 1H), 2.92 (q, 2H), 3.47-3.62 (m, 3H), 3.99-4.06 (m, 2H) ppm.

### EXAMPLE 6

### 7-Cyclopentyl-5-ethyl-2-(1-methoxy-1-methylethyl)imidazo[5,1-][1,2,4]triazin-4(3H)-one

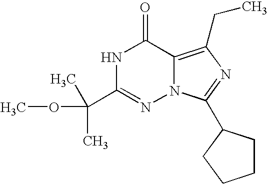

In analogy to the procedure for Example 1, 1.14 g (3.53 mmol) crude N-{1-[3-(1-methoxy-1-methylethyl)-5-oxo-4,5-dihydro-1,2,4-triazin-6-yl]propyl}cyclopentane-carboxamide and 1.08 g (7.07 mmol) phosphoric trichloride are stirred at reflux for 3 hours, proportionate amounts of the solvents are used.

Yield: 792 mg (74%)

¹H-NMR (300 MHz, CD₃OD): δ=1.26 (t, 3H), 1.54 (s, 6H, 1.65-2.17 (m, 8H), 2.95 (q, 2H), 3.21 (s, 3H), 3.52-3.65 (m, 1H) ppm.

### EXAMPLE 7

### cis-7-(4-tert-Butylcyclohexyl)-5-ethyl-2-(1-methoxy-1-methylethyl)imidazo[5,1 f]-[1,2,4]triazin-4 (3H)-one

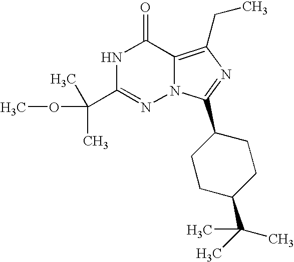

In analogy to the procedure for Example 1, 1.39 g (3.54 mmol) crude cis-4-tert-butyl-N-{1-[3-(1-methoxy-1-methylethyl)-5-oxo-4,5-dihydro-1,2,4-triazin-6-yl]propyl}-cyclohexanecarboxamide and 1.08 g (7.07 mmol) phosphoric trichloride are stirred at reflux for 3 hours, proportionate amounts of the solvents are used.

Yield: 485 mg (37%)

¹H-NMR (400 MHz, CD₃OD): δ=0.85 (s, 9H), 1.08-1.20 (m, 1H), 1.26 (t, 3H), 1.41-1.56 (m, 8H, s at 1.54), 1.59.1.67 (m, 2H), 1.68-1.79 (m, 2H), 2.33-2.41 (m, 2H), 2.96 (q, 2H), 3.21 (s, 3H), 3.43-3.49 (m, 1H) ppm.

### EXAMPLE 8

### cis/trans-5-Ethyl-2-(1-methoxy-1-methylethyl)-7-(4-methylcyclohexyl)imidazo[5,1-f][1,2,4]triazin-4(3H)-one

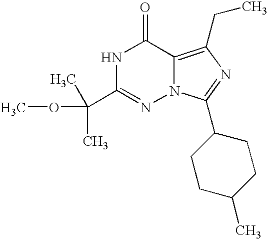

In analogy to the procedure for Example 1, 1.08 g (3.09 mmol) crude cis/trans-N-{1-[3-(1-methoxy-1-methylethyl)-5-oxo-4,5-dihydro-1,2,4-triazin-6-yl]propyl}-4-methylcyclohexanecarboxamide and 2.47 g (16.1 mmol) phosphoric trichloride are stirred at reflux for 3 hours, proportionate amounts of the solvents are used.

Yield: 1.01 g (98%)

¹H-NMR (300 MHz, CD₃OD, cis/trans mixture): δ 0.86-1.12 (2×d, 3H), 1.29-1.36 (m, 4H, t at 1.33), 1.57 (s, 6H), 1.61-2.17 (m, 8H), 3.01-3.10 (q, 2H), 3.24 (s, 3H), 3.32-3.36 (m, 1H) ppm.

### EXAMPLE 9

### cis-7-(4-tert-Butylcyclohexyl)-5-ethyl-2-(1-methoxycyclopropyl)imidazo[5,1-f]-[1,2,4]triazin-4 (3H)-one

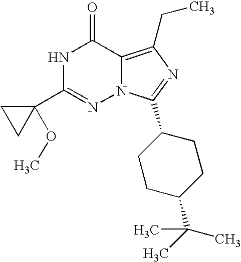

In analogy to the procedure for Example 1, 348 mg (0.89 mmol) crude 4-tert-butyl-N-{1-[3-(1-methoxycyclopropyl)-5-oxo-4,5-dihydro-1,2,4-triazin-6-yl]propyl}cyclohexanecarboxamide and 409 mg (2.67 mmol) phosphoric trichloride are stirred at reflux for 3 hours, proportionate amounts of the solvents are used.

Yield: 201 mg (61%)

¹H-NMR (CD₃OD, 300 z): δ 0.84 (s, 9H), 1.09-1.78 (m, 14H, t at 1.26), 2.26-2.36 (m, 2H), 2.96 (q, 2H), 3.34 (s, 3H), 3.37-3.43 (m, 1H) ppm.

### EXAMPLE 10

### 7-Cyclopentyl-5-ethyl-2-(1-methoxycyclopropyl)imidazo[5,1-f][1,2,4]triazin-4 (3H)-one

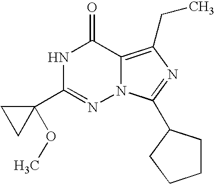

In analogy to the procedure for Example 1, 285 mg (0.89 mmol) crude N-{1-[3-(1-methoxycyclopropyl)-5-oxo-4,5-dihydro-1,2,4-triazin-6-yl]propyl} cyclopentane-carboxamide and 409 mg (2.67 mmol) phosphoric trichloride are stirred at reflux for 3 hours, proportionate amounts of the solvents are used.

Yield: 137 mg (51%)

¹H-NMR (CD₃OD; 200 MHz): δ=1.26 (t, 3H), 1.62-2.17 (m, 12H), 2.94 (q, 2H), 3.35 (s, 3H), 3.45-3.58 (m, 1H) ppm.

1. Compounds of the general formula (I) 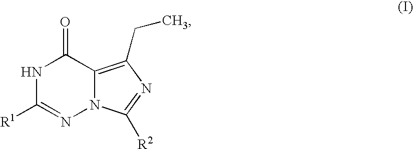
in which
R¹ denotes (C₅-C₈)-oxa-cycloalkyl, which can be substituted by 0, 1 or 2 residues independently selected from the group consisting of (C₁-C₆) alkyl and (C₁-C₆)-alkoxy, or
denotes (C₁-C₈)-alkyl or (C₃-C₈)-cycloalkyl, which are substituted by 1 or 2 identical or different (C₁-C₆)-alkoxy groups,
and
R² denotes (C₃-C₈)-cycloalkyl, which can be substituted by 0, 1 or 2 identical or different (C₁-C₆)-alkyl groups.

2. Compounds according to claim 1, whereby
R¹ has the meaning indicated in claim 1, and
R² denotes 4-tert.-butylcyclohex-1-yl.

3. Compounds according to claim 1, whereby
R¹ has the meaning indicated in claim 1, and
R² denotes cis-4-tert.-butylcyclohex-1-yl.

4. A process for the preparation of the compounds according to claim 1, characterized in that,
compounds of the general formula (IV), 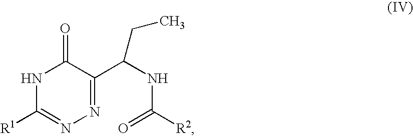
in which R¹ and R² have the meaning indicated in claim 1,
are reacted with a dehydrating agent.

5. Compounds of the general formula (IV) 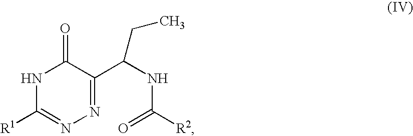
in which
R¹ denotes (C₅-C₈)-oxa-cycloalkyl, which can be substituted by 0, 1 or 2 residues independently selected from the group consisting of (C₁-C₆) alkyl and (C₁-C₆)-alkoxy, or
denotes (C₁-C₈)-alkyl or (C₃-C₈)-cycloalkyl, which are substituted by 1 or 2 identical or different (C₁-C₆)-alkoxy groups,
and
R² denotes (C₃-C₈)-cycloalkyl, which can be substituted by 0, 1 or 2 identical or different (C₁-C₆)-alkyl groups.

6. (canceled)

7. Pharmaceutical composition containing at least one compound according to any one of claims 1 to 3 and a pharmacologically acceptable diluent.

8. (canceled)

9. A method for the treatment and/or prophylaxis of inflammatory processes and/or immune diseases comprising administering to a human or animal an effective amount of a compound of claim 1.

10. A method for the treatment and/or prophylaxis of chronic obstructive pulmonary disease and/or asthma comprising administering to a human or animal an effective amount of a compound of claim 1.

11. (canceled)
